(19) United States
(12) Patent Application Publication
(10) Pub. No.: US 2025/0029699 A1
(43) Pub. Date: Jan. 23, 2025

(54) Method for Extracting Event Information from Calendar Representations

(71) Applicant: Arcascope Inc., Arlington, VA (US)

(72) Inventors: Olivia Walch, Falls Church, VA (US); Kevin Hannay, Comfort, TX (US)

(21) Appl. No.: 18/503,014

(22) Filed: Nov. 6, 2023

Related U.S. Application Data
(60) Provisional application No. 63/514,519, filed on Jul. 19, 2023.

(51) Int. Cl. G16H 20/10; G06Q 10/1093

(52) U.S. Cl. CPC G16H 20/10; G06Q 10/1097

(57) ABSTRACT

A method of extracting event information from calendar representations is provided. A calendar representation can be determined from an image of a calendar or schedule. A circadian state of a person might be determined by obtaining user data over a plurality of days, determining, for each of at least a subset of the plurality of days, an event type for each day, determining a circadian trajectory for a past day of the plurality of days, estimating a circadian trajectory and/or a circadian state for a future day and a mapping of the circadian trajectory and/or the circadian state to wall-clock time, and using the mapping to determine when to initiate or announce a biological treatment for the person based on a preference of circadian state for the biological treatment.

### CROSS-REFERENCES TO PRIORITY AND RELATED APPLICATIONS

This application is a non-provisional of, and claims the benefit of and priority from, U.S. Provisional Patent Application No. 63/514,519 filed Jul. 19, 2023, entitled “Method for Extracting Event Information from Calendar Representation.” The entire disclosure(s) of application(s)/patent(s) recited above is(are) hereby incorporated by reference, as if set forth in full in this document, for all purposes.

### FIELD

The present disclosure generally relates to methods of processing data and more particularly to methods of processing data to extract event information.

### BACKGROUND

It is long been known that the time of day a drug is taken can affect its efficacy and/or toxicity. More generally, a biological treatment applied to a human patient or another animal might be done at a particular time of day to maximize a desired outcome, such as efficacy, and minimize an undesired outcome, such as toxicity.

A biological treatment might be the administration of a drug, administration of a nutrient, administration of another substance, such as orally, intravenously, intramuscularly, cutaneously, or via other pathways. A biological treatment might be more than, or instead of, administering a substance. For example, a biological treatment might be the application of kidney dialysis, a surgical procedure, or another medical procedure.

Often, instructions about when to apply a treatment include time of day, such as “take one dose one hour before bedtime” or “take one dose every six hours starting at 8 AM.” The time-of-day provisos might be referred to as wall-clock time provisos. While those might be useful for someone who is operating on a daily schedule that aligns their internal circadian clock with time of day (wall-clock time), that might work well but not work so well for others. Humans and other animals can have internal states, circadian states, that vary over the course of a day or some other period that might be represented by, or a function of, a circadian trajectory corresponding to changes in those states over time. A circadian trajectory is not necessarily synchronized with the natural environment. For example, the circadian trajectories of a patient may be affected by changes in time zones during travel, changes from daylight savings time switches, changes in work schedule, shift work, stress, sleep schedules and the like.

Some biological treatments, such as exercise, light exposure, or an activity aimed at a physiological effect governed by the human circadian clock, have an effectiveness that can be highly dependent on timing the treatment based on the patient's circadian state along their circadian trajectory. The term “chronomedicine” and “chronomedical” might be used in the literature to refer to considering time of day effects of biological treatments and “chronomodulation” and “chronomodulated” might refer to scheduling the timing of biological treatments based on a circadian state and the process might be referred to as “chronotherapy.”

In some circumstances, time of day is a sufficient proxy for circadian state but in other circumstances it is not. In such cases, it can be important to be able to determine a current circadian state and when a particular future circadian state will be reached. For example, if a medication is known to be most effective when a patient's circadian state corresponds to an “early morning waking state” and because of the patient's work schedule, activities, travel, etc., that is determined to occur around 11:30 AM on a given day, the patient could be alerted in advance as a reminder to “take one dose as close as practical to 11:30 AM today.”

Determining a mapping between a circadian trajectory and the passage of wall-clock time can be done in some precisely controlled environments, such as a multi-day stay in a light-controlled room, blood monitoring, and other cumbersome and invasive testing, with results possibly not available for many days or weeks. As such, those measurement techniques are unwieldy and often timing of treatments are done as guesswork.

What might be needed for a patient treatment system that uses chronomedicine and chronomodulation are improved methods of determining time-related events for use in computing timing recommendations for patient actions.

### SUMMARY

A computer processing method for processing data about a user might determine that user's schedule with sufficient detail and/or resolution to be useful for a patient treatment system that uses chronomedicine and chronomodulation. The data might include raw images that include depictions of scheduling data, such as work schedule calendars, written events, and/or graphical representations of historical wearable data from which scheduling information can be extracted. The data might be derived from historical records to discern patterns for future schedules.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of methods and apparatus, as defined in the claims, is provided in the following written description of various embodiments of the disclosure and illustrated in the accompanying drawings.

### BRIEF DESCRIPTION OF THE DRAWINGS

Various embodiments in accordance with the present disclosure will be described with reference to the drawings, in which:

FIG. 1 illustrates an example of a trajectory having some uncertainty built in.

FIG. 2 is a diagram of an image processing system for extracting calendar event data from images.

FIG. 3 is a diagram of a patient treatment system, according to various embodiments.

FIG. 4 illustrates a plot of calendar activity as might be represented in a data structure readable by a patent interaction system and/or a patient treatment handling system.

FIG. 5 illustrates another plot of calendar activity as might be represented in a data structure readable by a patent interaction system and/or a patient treatment handling system.

FIG. 6 illustrates a collection of day-level activity collection as might be stored in memory for use by a patient system.

FIG. 7 illustrates an example calendar depiction that might be presented to a user or used in further processing.

FIG. 8 illustrates an example computer system memory structure as might be used in performing methods described herein, according to various embodiments.

FIG. 9 is a block diagram illustrating an example computer system upon which the systems illustrated in other figures may be implemented, according to various embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Examples of biological treatments might include administering a medicine to the patient, outputting a message to be received by the patient for them to take the medicine, and/or indicating an optimal time, or estimate thereof, for some other treatment. Where the treatment has a fixed wall-clock time, such as a surgery scheduled for November 18 at 2:00 PM, the patient treatment system could compute which inputs could affect the circadian trajectory and provide the patient with inputs, and/or administer some pre-surgery treatment, that would be expected to cause a shift in the patient's circadian trajectory such that a desired circadian state aligns with the fixed wall-clock time of the treatment.

In a patient treatment system, various inputs are obtained and possibly also precomputed machine-learning models and stored data are used, to determine, at least approximately, a circadian trajectory that maps between wall-clock time and a circadian state. The particular time of day might not always be aligned with “wall-clock” time. For example, someone who regularly works from 11:00 PM to 7:00 AM might have a circadian state corresponding to “early morning shortly after arising” at around 8:30 PM to 10:30 PM and a “ready to sleep” circadian state around 11:45 AM. The circadian trajectory might have varying degrees of uncertainty from patient to patient and/or from time to time for a given patient. A patient treatment system can determine, from a determined circadian trajectory and various circadian-mapping profiles for a given treatment, what wall-clock time, times, and/or time ranges, to administer a treatment. For such a patient treatment system, it can be important that the patient treatment system correctly determine, at least approximately, the patient's circadian trajectory.

One input that a patient treatment system might use to determine a patient's actual circadian trajectory is the patient's daily/monthly/etc. calendar, such as a work schedule calendar. Where such data is available in structured, computer-readable, data form, it can be a simple matter to have the patient treatment system ingest that data. However, it is often the case that work schedules and other calendar data is only available in other forms. For example, workers might only be provided access to a printed and posted schedule announcement and then might only be able to take a photo of the posted schedule announcement on their mobile phone. In some cases, the schedule is not explicitly provided and might need to be derived from historical data, such as a user's historical activity pattern.

It may also be the case that the worker has no representation of the schedule that can be readily ingested by a patient treatment system. For instance, the worker may be called in for an unexpected work shift via a text message, without ever having a representation that could be recognized as a calendar event. In combination with data about a patient's schedule, travels, and other data that might be recorded in their electronic calendar, data about past activity patterns and other patterns might be used to determine or refine a circadian trajectory and/or determine relative uncertainties as to the circadian trajectory.

In a patient treatment system, various inputs might be obtained, such as wearable device data from a wearable device worn by a patient, other sensor data from the patient, calendar data from a calendar program of the patient, location, travel, environmental, etc. inputs. In some cases, the device might be attached to the user, attached to or part of the user's clothing, placed in the user's pocket, bag, etc., or other methods of having the device pick up user movements and/or activity.

From some or all of those inputs, as explained herein, and possibly also from precomputed machine-learning models and stored data that is not necessarily specific to that patient, a circadian trajectory that maps between wall-clock time and a circadian state is determined. The circadian trajectory might have varying degrees of uncertainty from patient to patient and/or from time to time for a given patient, such as when fewer inputs are available, inputs are inconsistent, and/or for other reasons. The patient treatment system can determine, from the circadian trajectory and circadian-mapping profiles for a given treatment, what wall-clock time, times, and/or time ranges, to administer a treatment.

The time, or time ranges, for administering a treatment or signaling for the administration to occur, might be determined based on circadian-mapping profiles that indicate, for example, what circadian states relate to high efficacy and/or low toxicity. In a specific example, if a patient's circadian state at a snapshot in time is represented in memory by a value between 0.0 and 1.0 and the patient treatment system determines that efficacy for a drug D is highest at a state value of CS=0.4 and toxicity of drug D is lowest at that state value as well, and further that the patient treatment system determines that CS=0.4 corresponds to 10 AM with an uncertainty of three minutes, the patient treatment system might send a message to the patient, perhaps on a wearable device and perhaps some time in advance, to the effect of “Please take your pill D sometime between 9:57 and 10:03 AM for best results.”

Other scales are possible for circadian state, such as circadian state being considered like a circular phase and continuously varying from 0 through to 2*π, which the patient treatment system could consider to be the same as 0. A circadian trajectory can represent past circadian state changes as a function of wall-clock time and/or anticipated or predicted future circadian state changes as a function of wall-clock time. Wall-clock time could be represented by a clock circuit, a computer circuit that keeps time, and/or a computer element that receives signals representing a time measured independent of bodily activity or details such as a local time, which might have varying resolutions, such as from {morning, daytime, noon, afternoon, evening, late night}, to HH o'clock, to HH:MM on day DD, to HH:MM:SS on day DD of month M in year Y.

Wall-clock time can be represented in a number of ways, such as a stored value of between 0.0 and 1.0 representing a time of day, perhaps in resolutions of seconds or some other interval, a stored value of between 0:00 and 23:59:59 representing a time of day, an epoch time, such as a number of elapsed seconds since some specified time and date. As an example of epoch time, a stored time value, t, where t=1642724939 might represent a wall-clock time of 12:28:59 AM UTC on Jan. 4, 2022.

In some instances, a patient treatment system might maintain multiple circadian trajectories for a patient and use one or more of those maintained circadian trajectories for particular treatment management. Examples of multiple maintained circadian trajectories might include a central circadian trajectory that maps a body's central circadian state to wall-clock time where the central circadian state corresponds to the body's suprachiasmatic nucleus (SCN), such as a representation in memory of instantaneous firing patterns in the suprachiasmatic nucleus, corresponding to how neuronal firing patterns change over time, and/or patterns in changes of concentration of clock genes within cells.

A peripheral circadian state might reflect circadian-relevant molecular concentrations in a body and a peripheral circadian trajectory representing changes in that peripheral circadian state over a period of wall-clock time. For example, one particular peripheral circadian state might represent a current concentration of sodium-proton exchanger (NHE3) in the intestinal lumen of a patient. Computing circadian states using patient data and less invasive body sensors (e.g., heart rate monitors, movement monitors) would be easier than directly measuring state by, for example, having an internal sensor continuously measuring NHE3 concentration in the intestinal lumen.

Concentrations of molecules that rise and fall with a circadian pattern can be described by circadian trajectories. These trajectories might be called “peripheral circadian trajectories” when they occur in peripheral clocks, such as the biological clock mechanisms related to the stomach or the liver. These trajectories might be called “central circadian trajectories” when they occur in the central clock, the SCN. These trajectories, or estimates thereof, can be generated using digital twin simulations. In one instance, digital twin simulations of circadian trajectories are generated by a processor according to a differential equation describing the concentration of the molecule, enabling the concentration to rise and fall in a dynamical, physiological manner.

Determining the circadian trajectory can be a complicated process, since some input data might be missing and/or there might be some disruptions in the patient's normal schedule, such as travel over several time zones. In some cases, a circadian trajectory might need to be computed on the fly, in real time, or near real time and therefore some optimizations in the processing of data might be required. Furthermore, some of the computations might be done on a low-powered device. In some implementations, part of the computation is done remotely, perhaps on more powerful servers, and part of the computation is done on a low-power, local, wearable and/or portable device with communications capability.

In a typical configuration, a circadian trajectory is continuous in that (1) two nearby points on a circadian trajectory map to nearby wall-clock times, and (2) a circadian state at the end of the circadian trajectory is the same as, or close to, the circadian state at the beginning of the circadian trajectory such that a patient treatment system does not experience a discontinuity in circadian state from day to day or cycle to cycle. While a circadian trajectory could map exactly to wall-clock time, e.g., a linear mapping, more typically a circadian trajectory will compress or expand units of circadian time relative to wall-clock time, which might be treated as a circadian clock and a wall clock running at different rates and the ratio of the different rates varying over a day or other measurement period. In general, a complete circadian trajectory can provide a mapping from a circadian state to a wall-clock time as well as providing a mapping from a wall-clock time to a circadian state.

Whereas some treatments might be prescribed strictly on wall-clock times (e.g., “take one pill early in the morning and a second one at noon local time”), other treatments might rely on circadian time that represents some circadian state on a circadian trajectory. Such other treatments might benefit a patient more if that circadian trajectory and/or a current circadian state is more accurately and/or precisely determined.

The circadian trajectory can be represented and stored in computer memory as a lookup-table, possibly corresponding to a piecewise linear plot of circadian time versus wall-clock time. The patient treatment system might store the circadian trajectory as a vector data structure that can be manipulated by computer instructions created for performing vector operations or hardware capable of vector operations. The resolution of such a plot of circadian time versus wall-clock time might be on the order of seconds or minutes. For example, a 24-hour plot of circadian time versus wall-clock time might be stored as a vector of 288 values each corresponding to a circadian state at five-minute intervals.

A patient's body has biological states that might vary over time. For example, a stored biological state value might reflect a concentration of melatonin in the patient's saliva, the concentration of caffeine in the patient's bloodstream, the concentration of the Bmal1 gene in the patient's brain, the current firing rate in the patient's suprachiasmatic nucleus, etc. For nonhuman patients, some biological states might not have human counterparts. One particular biological state is a circadian state.

A patient's circadian state is a biological state corresponding to where the patient is in a circadian cycle. As explained herein, a given body might have multiple circadian cycles, not all of which need be aligned, such as a central circadian cycle and peripheral circadian cycles. Melatonin concentrations, Bmal1 gene concentrations, firing rate of the SCN, and other biological states might be circadian states or proxies therefore.

A time sequence of a biological state can be stored and/or represented in memory as a time series of state values, or a trajectory for the biological state. The representation of a trajectory in computer memory can be in the form of a listing of records each indicating a state value and a time, a piecewise linear plot, coefficients of a fitted curve, or other form of data structure usable for representing state values and specified times.

An example biological state might be that a melatonin concentration is 4.3 picograms/ml at epoch time t=1642724939. Another example biological state might be that core body temperature is 97.3° F. at epoch time t=1642724939. A value, R, might be stored in memory as a representation of some unitless or unit-specific quantity corresponding to a biological state. For example, it might be a value of a biological oscillator such as melatonin concentration in saliva in units picogram per milliliter. Another example is a unitless quantity corresponding to a measure of cohesion of neurons firing in a suprachiasmatic nucleus and might be such that R=0 represents a state wherein the neurons are completely out of sync with each other and R=1 represents a state wherein the neurons are firing perfectly in sync with each other.

As an example of a circadian state, it might be represented as a sinusoidal curve defined by having an amplitude of Rₐ=0.4 and at an epoch time of t=1642724939 having a phase of Ψ=π/4. A period of a trajectory, which might correspond with a circadian cycle might not be constant and can change from period to period (e.g., a 24.75 hour circadian cycle followed by a 25.4 hour circadian cycle then followed by a 24.9 hour circadian cycle. The effective period of a trajectory, the rate of change of the state might also change dynamically over the course of a trajectory as time shrinks and expands.

In some cases, an intrinsic period might be present and/or measured, which might correspond to a circadian cycle that a body experiences in the absence external time-marking signals, such as light variations throughout the day. A patient treatment system might store a value for the intrinsic period that can be used as a parameter that can be tuned to improve estimates of a person's circadian state, along with other parameters that might really matter to phenotype, like light sensitivity, in defining a trajectory. Varying such parameters can be used to generate a trajectory bundle, and the range of parameters can be narrowed or made wider if other information about them is available, such as by giving a person a test to see how much their pupil constricts to assess light sensitivity.

In another example, a circadian state might be stored as a representation of multiple gene expressions, such as a vector value [32.242, 484.23, 4994.2 . . . ] for t=16427249 representing expression levels for genes [Gene₁, Gene₂, Gene₃, . . . ]. For some such circadian states, there might be hundreds of genes represented.

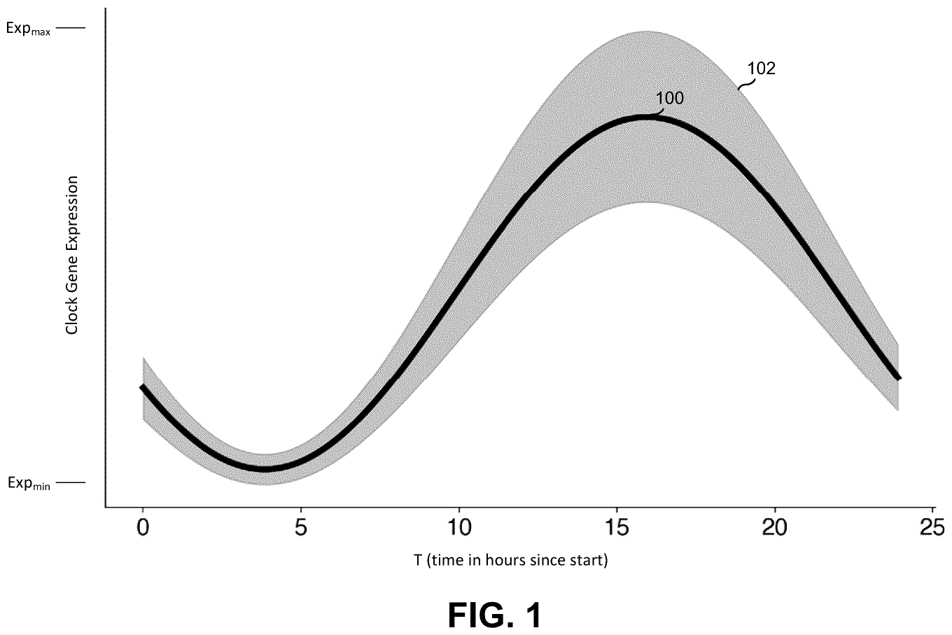

FIG. 1 is an illustration of a trajectory and illustrates an example of a trajectory 100 having some uncertainty 102 built in. Uncertainty for each state in a trajectory can be calculated with a trajectory bundle. The spread in the trajectories at a given time can be used to define the uncertainty for each circadian state. As stored in memory, a trajectory might be represented by a table of tuples (wall-clock time, circadian state, uncertainty measure) at some resolution (e.g., one tuple per five minute span, one tuple per 15 minute span, one tuple per hour) and each circadian state might be a value such as a phase that sweeps from 0 to 2π mod 2π or from 0 to 1 mod 1, or a metric such as an estimated concentration of an internal substance that has a concentration that follows a circadian rhythm or a clock gene expression rate. For simplicity of explanation, in many examples, some normalized smooth curve might be illustrated with the understanding that circadian trajectories might not be so smooth and predictable. An uncertainty measure for a given tuple might be a percentage or a quantity.

Uncertainty might be due to wide variances among trajectories. For example, where a patient treatment system might have trajectories for several different input values, but lacks the input value or certainty over the input value, the trajectory might be uncertain, as reflected in the plot shown in FIG. 1. Uncertainty might relate to, for each circadian state, central or peripheral, a reflection of how confident an estimation of that state is. For example, at time tₓ, a concentration of a particular molecule could be between 3 and 4 picograms/ml and that variance might contribute some level of uncertainty for time tₓ. On the other hand, if the concentration were only known to be between 3 and 300 picograms/ml at time tₓ, that would be a higher uncertainty. Circadian uncertainty might increase in light of work or other schedules. Uncertainty can increase in the presence of missing data and can decrease in the presences of large volumes of input data, so having more data might reduce the uncertainty.

Circadian-mapping profiles that map efficacy, toxicity, etc. variances to circadian state can be developed by collecting examples from a large number of patients and processing that data into circadian-mapping profiles. These circadian-mapping profiles can be stored as data structures that are processable by the patient treatment system and might be used with trajectory data to make treatment timing decisions and/or recommendations.

A treatment mapping can be represented in a data structure that provides a rule for converting a circadian trajectory with affiliated uncertainty and a circadian-mapping profile into a set of administration windows. An administration window can be narrower (e.g., “take a drug between 5:00 PM and 5:30 PM”) when the uncertainty is low. An administration window can be wider (e.g., “take a drug in the afternoon”) when the uncertainty is high. The treatment mapping can be computed from a patient's circadian trajectory, which in turn can be derived from wearable data. For instance, wearable data can be used to arrive at both a circadian trajectory and uncertainties for each point by simulating a digital twin of the suprachiasmatic nucleus (SCN). In this example, a circadian state can be represented by the firing rate and firing cohesion of the neurons in the SCN, or by the expression of genes in SCN cells in a moment of time.

A patient management system or patient control system might take into account a current circadian state, with affiliated uncertainty, as well as a fixed treatment window, and prescribe a set of controls to move a recommended administration window, which corresponds to a future circadian trajectory and an efficacy and/or toxicity profile, to overlap as much as possible with the fixed treatment window.

Accurately determining an individual patient's circadian time so that it can be used in the treatment mapping might include wearable data augmented by a data structure such as a work calendar data table. Inclusion of this data structure can reduce uncertainty.

Knowledge of calendar events can reduce uncertainty around circadian state by augmenting other signal data about a user, such as that a user was awake during some period, as well as what circadian relevant-stimuli they were likely to be exposed to during that period. In addition, this knowledge can help uncover if the user's circadian state was likely to have been disrupted by recent travel or work. Chronic disruption, such as regular shift work or travel, can also provide information that can be used to tune or adapt the tracking of circadian state (e.g., if a person is a long-term shift worker, they may be experiencing long-lasting changes to their circadian rhythms, which show up as changes to their circadian state), and this in turn can reduce the uncertainty of a tracked circadian state.

A patient management system or treatment system might use calendar event extraction to aid in the identification of anomalous days or events which are likely to introduce short-term disruption. Knowledge of calendar anomalies can also assist in reducing uncertainty in predictions of circadian state, which can in turn alter the recommended treatment windows.

The administration windows may be presented as only timing recommendations for doses with a fixed amount, such as pills (e.g., “there is a good time of day to take this pill and for you specifically, that time of day is between around 10 AM to 11 AM local time tomorrow”), or they can be presented as both dosing and timing recommendations (e.g., “your morning dose should be X mg between around 10 AM to 11 AM local time and X+Y mg between around 12 PM and 1 PM”).

Inputs to a circadian-mapping profile can include known optimal timings for the individual based on self-report or other records, a set of timing restrictions represented in a restrictions table or stored restrictions rule set (e.g., restrictions on time intervals between doses), and the like.

A user interface might be provided to inform the patient of administration windows and alerts at wall-clock times that correspond to administration windows.

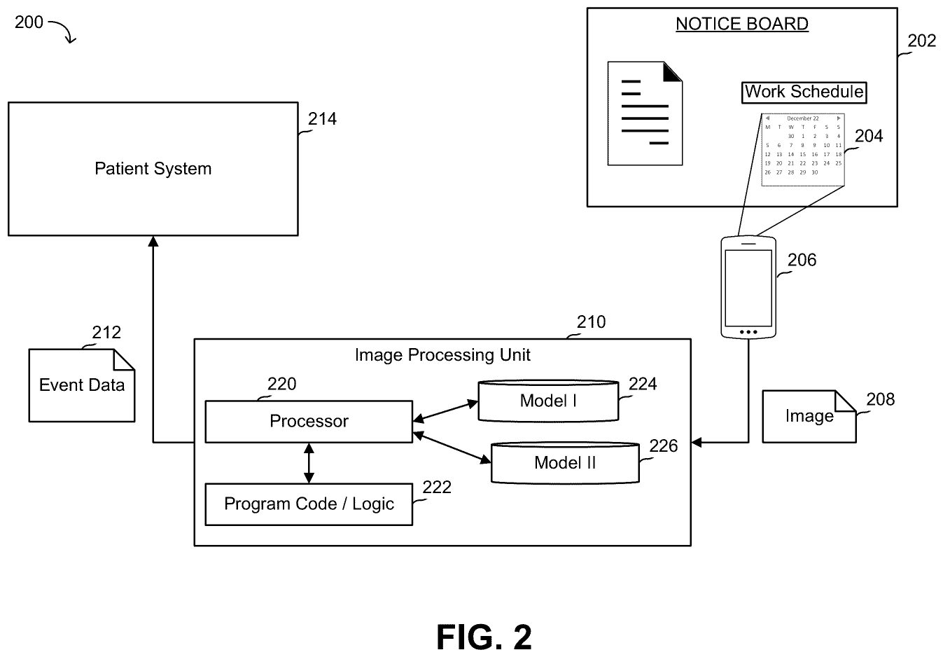

FIG. 2 is a diagram of an image processing system 200 for extracting calendar event data from images that might be used to process and/or estimate a work schedule that could in turn be an input to a circadian mapping system. In the example shown there, a work schedule 204 is presented on a notice board 202 and a worker might capture an image of work schedule 204 and/or notice board 202 using, for example a smartphone 206 equipped with a camera to generate an image 208. Image 208 can then be input to an image processing unit 210. A work schedule might be obtained in other ways, such as being sent by text messaging, available in a transmitted image, an e-mail, etc. possibly intermixed with other elements.

Image processing unit 210 then processes image 208 to determine event data 212 which can then be sent to a patient system 214 so that patient system 214 can use event data 212 as an input to processing to determine a circadian trajectory. Image processing unit 210 might comprise a processor 220, program code and/or logic 222, and one or more machine learning models, such as model 224 and model 226.

A machine learning model might be an output of a training system that trains on known data to be able to match unknown data. The training system might train a model using image-to-text machine learning, wherein text produced is in a universal calendar format. Given that calendar data is often highly structured, a large quantity of synthetic data could be easily generated. For example, a training system might include a synthetic data generator that generates billions of fake calendar images representing different months, different ways of representing events, different affine transformations, different lighting conditions, different incidences of missing or obscured data, etc. The accompanying ground truths for those images would be known since the images themselves are synthetic and generated. This could also be done in a hybrid manner, with grid detection methods from computer vision paired with image-to-text models operating on segments of the grid. Once trained, a model can be used in evaluating an image of a calendar where no ground truth is known a priori to determine what the calendar data is in the image.

### Image Processing to Extract Event Data

The input to an image processing unit might be an image, and an output is a data record, message, and/or other computer-readable event record. The event record might have a data field indicative of a start date, an end date, a start time, an end time, a start time plus a duration, etc., and possibly a categorization and a textual event descriptor. Some events might represent scheduled work shifts or meetings, and some events might represent “negative” events, such as those dates that are “off-days” for the user of the output of the image processing unit. The textual event descriptor might be blank, might be generic (e.g., “Event”), as alleged or might be blank.

The image input to the image processing system might include a calendar representation, such as a screenshot of a digital calendar, a photo of a display calendar, and/or the like. In some images, a calendar might be a representation of a historical time series of data, segmented by day or other time unit. For instance, it might be an image of historical activity patterns, where each row in the image comprises step counts over the course of a day and each column corresponds to a time of day. A calendar representation might cover a single day, or it might cover multiple days. For example, it might be one week, or one month, or several months. Durations in the calendar might be represented without spatial information corresponding to their length. For instance, events that are three hours might take the same space as events that are one hour. Durations in the calendar might also be represented along an axis, in which their duration is represented by a size of the event's representation within the calendar. For instance, a three-hour meeting might be three times as tall as a one-hour meeting.

Events within the calendar might be represented as short pieces of text containing event descriptors and date/time instances. For instance, an event might be represented as “Shift from 11:00 pm to 3:00 am.” An event might be represented without an explicit descriptor. For instance, an event might be represented by the string “7:00 pm-11:00 pm.” In this case, the event might be described with a placeholder descriptor, e.g., “Shift.”

An event might be represented with just a start time, or just a location along an axis, and the duration might be inferred to be a fixed value or to be set by the axis of the calendar representation. For instance, an axis might be used to infer how long a meeting goes even if there is no end time. It might also be used to infer when the start time is even when there is no start time explicitly attached to the event.

An event might be represented pictorially, as an icon, color, or combination of icon and color. Events might be handwritten. The calendar representation might have a grid with grid lines separating the days. The calendar representation might also have no grid lines. The calendar representation might include the month and year for the dates being display or it might not. Given all the variations, the image processing system might need to be trained on calendars to be able to extract and understand calendars enough to be able to extract event information from an image of a calendar representation.

As explained above, a training system can generate training data synthetically, given the known structure of calendar representations. Billions of images with programmatically assigned events, times, languages, angles of view (affine transformation), lighting conditions, and obfuscations can be generated by code. These images can in turn be used to train a model for extracting calendar information as text from non-synthetic, actual images in the wild. Hybrid approaches, which combine grid detection with image-to-text machine learning models trained on billions of images, could also be used to improve the training process, by breaking it into two steps: first, identify a grid; second, extract information from the grid according to rules from the training dataset.

Once event data is determined from calendar representations, the events represented by that event data might be added to a digital calendar format message, such as a standardized, text-based object. An example method of processing might include identifying the type of calendar, identifying a grid structure within the calendar, segmenting the calendar into day rectangles or zones of an image, extracting either date/time instances or date clusters from the days, converting date clusters into date/time events into using alternate sources of information, such as alternative calendar representations, and adding missing information, such as month or year, using alternate sources of information, such as alternative calendar representations.

The method might include identifying a calendar grid in the absence of grid lines. This method might include (1) digit or date identification with optical character recognition (OCR), and (2) grid imputation, using the identified dates or digits, with a method robust to incorrectly recognized dates or digits. Grids might be imputed using a voting method, in which every identified date or digit “votes” for the calendars it might be a part of. Grids might be imputed by overlaying a template grid and performing a gradient descent search to stretch and size it to the digits. These two methods might be combined.

The method might include extracting events from a day region. This method might include identifying text within the image, identifying whether the text in the image contains both a start and end time or just a start time, or neither, identifying an axis within an image and, if one exists, using the axis to determine start time and/or duration of an event.

Identification of an axis within an image might include calculating pairwise distances between all identified date/time instances and selecting the dominant mapping of pixel distance (vertical or horizontal) to time duration according to some rule. Identification of an axis within an image might include taking horizontal or vertical slices of the image in order to ascertain when a pixel transition happens, marking the end and start of an event.

Date clusters might be extracted from a day rectangle. This method might be done by (1) Identification of clusters, using supervised or unsupervised learning, and (2) Completing the information in the clusters using alternate data sources. Alternate data sources might be user-driven; e.g., the image processing unit might interact with the user to ask the user what hours an event marked with an icon corresponds to. Alternate data sources might also be calendar representations themselves, such as historical pattern data, e.g., historical wearable data. The image processing unit might process a user's historical activity data and look for patterns that match the patterns in the calendar. For instance, if image processing unit sees two days in one cluster followed by two days of another in the calendar representation, it might infer two day shifts followed by two night shifts in the wearable data and either select a mapping of cluster 1 to day shift and cluster 2 to night shift, or might present it to the user to confirm.

Some calendar processing systems can look at prior calendar representations to determine a predicted future pattern. For example, if a calendar processing system has data indicating that a user works the night shift from 10 PM to 6 AM five nights in a row, followed by three days off in a row, the calendar processing system might infer that that pattern would repeat for future days. That prior data that is used for predicting future patterns might be calendar representations, wearable device data, or other sources. Thus, a calendar processing system might have an image processing unit to obtain calendar data from captured or received images but might also have an inference unit that predicts or estimates future calendar data from past data. The inference unit might process a user's historical activity data and look for patterns that match the patterns in the calendar. For instance, if image processing unit sees two days in one cluster followed by two days of another in the calendar representation, it might infer two day shifts followed by two night shifts in the wearable data and either select a mapping of cluster 1 to day shift and cluster 2 to night shift, or might present it to the user to confirm.

Historical pattern data could also be other streams of data, such as time series of device usage, GPS data, heart rate data, etc.

One component of a method might add missing data to an extracted time using alternate calendar representations. For instance, the image processing unit might know the start and end time of an event but not which month and year it corresponds to. One way this might be done is by looking at nearby months to the current time and seeking those that match the calendar under consideration. For instance, if the image processing unit knows that the calendar says “November,” and the current year is 2023, the image processing unit might assume the calendar corresponds to November 2023. Similarly, if the image processing unit knows the calendar starts with Monday as the first day of the month, it has 31 days, and the current date is September 2022, the image processing unit might assume the month is August 2022, because Aug. 1, 2022 was a Monday.

The image processing unit might use historical pattern data. Historical pattern data might be searched through to find patterns matching those observed in a calendar. For instance, the image processing unit might could look at someone's wearable data and note that they were active on 3:00 am-7:00 pm on Aug. 8, 2022 and active from 7 am-7 pm on Aug. 11, 2022. From this, the image processing unit might decide that the calendar with events from 3:00 am-7:00 pm on the 8th day of the month and events from 7:00 am-7:00 pm on the 11th correspond to the month of August 2022, even if that image is missing from the representation.

A calendar representation that is historical pattern data, segmented by days, might be used to extract event information. This event information could be the presence of a work shift, or it could be the presence of an alarm going off, or it could simply be the presence of historical activity.

In one embodiment, presence of an alarm going off might be identified by the clustering of historical pattern onsets very near a round minute value. For instance, if activity onsets on a number of days occur a few seconds after 7:02, an event with an alarm going off at 7:02 might be inferred, as it is unlikely it would happen by chance, and it is generally not possible to set an alarm to the seconds level.

An event might be extracted from a calendar representation that is historical pattern data similar to how event information was extracted above using an axis: by taking horizontal or vertical slices of the image to calculate transition points wherein a background color or other representation of activity changes.

Events might be extracted from calendar representations that are historical pattern data using clustering. For instance, an autoencoder might be used to cluster historical pattern days to identify different types of days. These types of days might be on days, off days, transitions days, or the days might correspond to a specific type of shift.

Extracted events from a calendar representation might be used to identify patterns in events. For instance, a pattern might be “Work nights Monday through Friday” or “Work Two Days on Night, Two Days on Days, Four Days Off.”

Alternate calendar representations, including historical pattern data, might be used to update events in case an event in the calendar does not actually occur. For instance, an image calendar representation might say that an event occurs from 7:00 am-7:00 pm, but a historical pattern data calendar representation might suggest that the person was out sick that day. In this event, the event from the first representation might be removed or annotated to show that it might not have happened.

The pattern matching can also be used to estimate when people are sick or otherwise absent from normally planned events and might be used to estimate precursors of when people are sick or otherwise absent from normally planned events. For instance, a particularly challenging shift schedule might lead to more cases where two calendar representations diverge, suggesting absenteeism. In this case, the particularly challenging shift schedule could be identified, and recommendations could be made to a user or their employer to avoid this shift.

The image processing unit might be used to understand what shift combinations work best for different individuals, possibly in combination with the patient system. For instance, someone who is a night owl might show reduced calendar representation divergence suggesting absenteeism on a string of night shifts than someone who is an earlier type. This method might be used to make suggestions to the user for what shifts they should seek and avoid. This method might also be used for other outcomes, not just absenteeism. For instance, the absence of sleep might be noted if events take the entire day; for instance, if an event extracted from a historical wearable pattern shows activity for nearly 24 hours. The precursors to a day with a pronounced absence of sleep might be identified, and recommendations might be made. For instance, a worker who does not appear to sleep when transitioning from a day shift to a night shift might be recommended to adapt to a different schedule in which they are better able to sleep. Another outcome of interest could be resting heart rate or mood.

### Variations

Environmental controls can also integrate with a control system. For example, a control system may interact with environmental controls to change an environment based on a set of rules or data that move future administration windows to overlap as much as possible with fixed treatment times. As an example, the environmental control system might alter light levels, light colors, heating, cooling, etc. in ways that would alter a patient's circadian trajectory so that the resulting treatment mapping recommends an administration window near or overlapping a fixed treatment time.

A treatment system can gather inputs, apply a treatment mapping, and provide a user interface to indicate, possibly at or near certain wall-clock times, computed administration windows. The administration windows may initially cover long windows of time (e.g., “all afternoon”) and then become more specific as uncertainty is reduced (“between 8 and 8:30 PM”).

As described herein, a treatment system can take in patient inputs, and other inputs, process a treatment mapping that intakes biological states and at least one circadian state, along with a circadian-mapping profile, producing administration windows which correspond to wall-clock times. The display of the administration windows can be triggered near the time of the start of the administration window, and this display can be provided through a user interface that would issue a treatment administration message, such as a reminder to a patient or caregiver to administer the corresponding treatment.

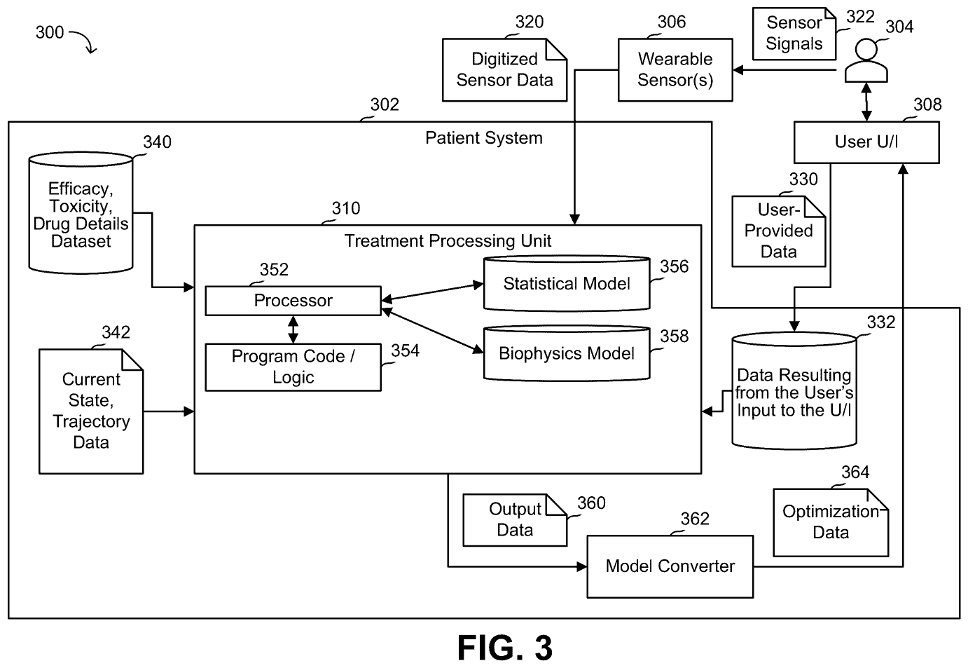

FIG. 3 is a diagram of a treatment system 300, according to various embodiments. Treatment system 300 can be used for producing optimal administration windows related to a biological process. In one example of the treatment mapping to determine best timing, a best time for taking a drug can be seven hours after the circadian state corresponding to melatonin onset. In another example, the treatment mapping can include a complicated molecular model that has equations capturing how different molecules in the drug bind to the body and interact with each other in order to select the best administration window given these drug interactions. In another example, the treatment mapping can take demographics information such as ethnicity, sex, age, and other drugs as parameters for input. Logical gating may be used and in one example, the treatment mapping ensures drugs are spaced at least twenty-four hours apart. In a further example, where molecules bind and interact in the equations, the variables represent molecular equations and parameters represent binding rates.

The information provided to the treatment mapping can include data on pill consumption, such as a pill that automatically tracks that it has been collected, or detection of wrist motion via an accelerometer that is predicted to match the wrist motion of taking a pill. In this example, the treatment mapping can use the pill timing information to arrive at more accurate trajectories for the pill concentration in the body. It can also use this information to apply logical gating rules, such as spacing between the intake of two consecutive doses.

As illustrated in FIG. 3, treatment system 300 might include a patient system 302 that interfaces with a user 304 via wearable sensors 306 and/or a user interface 308. Patient system 302 is shown comprising a treatment processing unit 310 that receives digitized sensor data 320 from wearable sensors 306 where the sensor data is derived from sensor signals 322. Sensor signals 322 might provide user data such as heart rate, movement, and other bodily indicators. User interface 308 can be used to obtain user-provided data 330 from user 304 which can then be stored in a data table 332 for use by treatment processing unit 310. User-provided data 330 might include demographics data, work schedules, travel information, drug information, and other information obtained from the user and/or from electronic devices of the user, such as a user's smartphone that might contain the user's calendar.

In operation, treatment processing unit 310 can read data from a drug dataset 340 that contains information about efficacy, toxicity, and other details of various drugs/medications possibly including time-dependent effects of various medications. Treatment processing unit 310 also takes in current state/trajectory data that might include interaction information from molecular stimulations. Treatment processing unit 310 can include a processor 352 that executes program code from program code storage 354 and operate using a statistical model 356 and a biophysics model 358.

A biophysics training system might generate a biophysics model, such as biophysics model 358, using human experimental data to represent the physics of the human body, such as modeling the way human physiology responds to stimuli. This might be based on data from experiments as well as inferred properties of dynamical systems. For instance, a biophysics model of the human circadian clock may capture the way the human body responds to an impulse of light conveyed via the retinohypothalamic tract by altering the voltage and resulting firing rates in the ventral and dorsal suprachiasmatic nucleus. In such a model, there could be a term or terms representing the retinohypothalamic tract (e.g., R), the ventral SCN (e.g., SCN_v), or the dorsal SCN (e.g., SCN_d).

A statistical model generator might generate a statistical model, such as statistical model 356, wherein probability are assigned for some output based on a given input, given training data. The statistical model could identify the most likely time dim light melatonin onset, or DLMO, will occur from a wearable time series. Statistical models and biophysics models can be combined. For instance, a statistical model can use wearable data that has already been processed by a biophysics model as its input. For instance, a statistical model could be trained on a dimensionality reduction of light data in which light data is fed into a model of the retinohypothalamic tract, and then reduced into a prediction of firing rates in the ventral and dorsal SCN. In this example, the training data for the statistical model could be the predicted firing rates (SCN_d and SCN_v) taken from the biophysics model.

Treatment processing unit 310 can determine, from digitized sensor data 320, drug dataset 340, and/or current state/trajectory data 342, a best time or time range for application of a treatment such as the taking of a medication wherein output data 360 is relative to a circadian clock. A model converter 362 might convert data as needed to wall-clock time and provide optimization data 364 to user 304 via user interface 308. Optimization data 364 might indicate a best time or time range for applying the treatment for that particular user.

A model converter might just take output data and change, for each action specified in the output data, the circadian states to wall-clock times. In some other examples, the model converter might determine timing of dim light melatonin onset (DLMO) on a given day, determine timing of predicted concentration of cortisol over a day or multiple days, determine predicted firing rates in a suprachiasmatic nucleus over a day or multiple days, determine predicted interactions between multiple drugs and the circadian clock over days or multiple days, or similar outputs.

A model converter might be considered a module that maps circadian state and trajectory onto a timeline, and can include steps of extract a sense of “time” from the model output (e.g., finding peak cortisol concentration and outputting some corresponding instruction for that event) and/or applying some rules for the best time for an action (e.g., in the case of the drug interactions, when to take a drug).

In some embodiments, calendar data is represented by an activity level value per time unit. While the calendar data might have been derived from an image of a calendar or smartphone calendar app data with additional information (e.g., not just that the user was awake and busy from 1:00 AM to 1:15 AM but that they were working on the night shift during that period at a particular street address), it can be sufficient to use data where only a single scalar value is provided for each time unit.

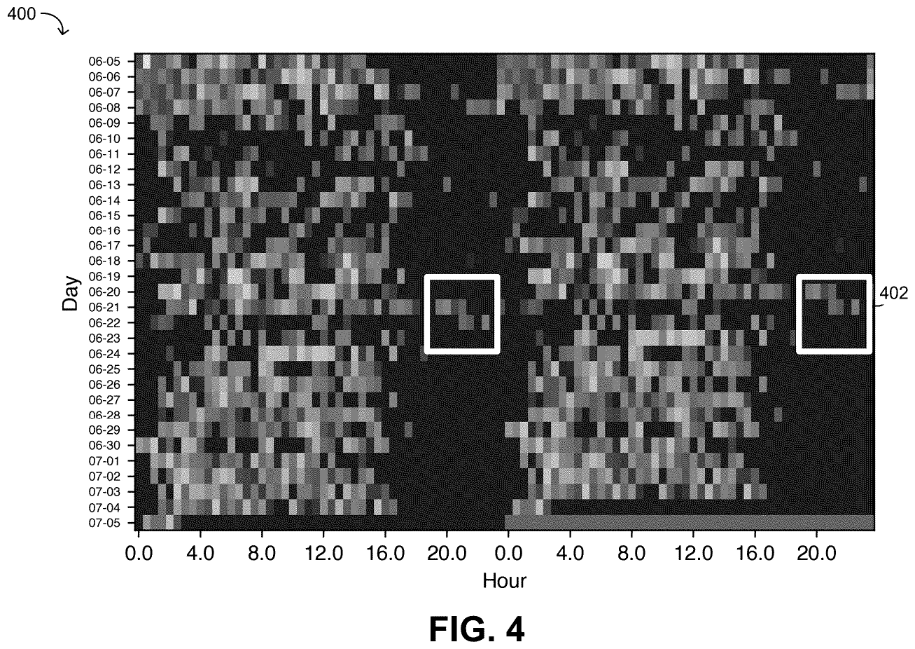

FIG. 4 illustrates a plot of calendar activity 400 as might be represented in a data structure readable by a patent interaction system and/or a patient treatment handling system where the calendar data is distilled down to a scalar value per time period. In FIG. 4, calendar activity 400 is graphically depicted by shaded rectangles, with each shaded rectangle representing an activity level of the user in a 30-minute interval with each row of the depiction showing a two-day span (e.g., time on the x-axis is double plotted). In this example graphical calendar representation, a lighter gray or white rectangle represents a 30-minute interval with more activity and a darker gray or block rectangle represents less activity. The activity levels might be values that correspond to the amount of activity detected by sensors, which might be sensors of a wearable device or a device that the user carries.

As can be readily seen from the depiction of the calendar activity, the user is less active in the intervals between 16.0 and 0.0 (which is 24.0). Note that 0.0 on the calendar depiction does not necessarily coincide with the user's 12 midnight wall-clock time. As part of a patient system, a processor can analyze the data and flag anomalies, such as an anomaly 402 identified in the white box. (It appears twice because time on the x-axis is double plotted.) This anomaly might represent an intraday calendar event, such as a late night. An event capturing the main activity period over the day can be extracted at the day-level, represented as a date cluster.

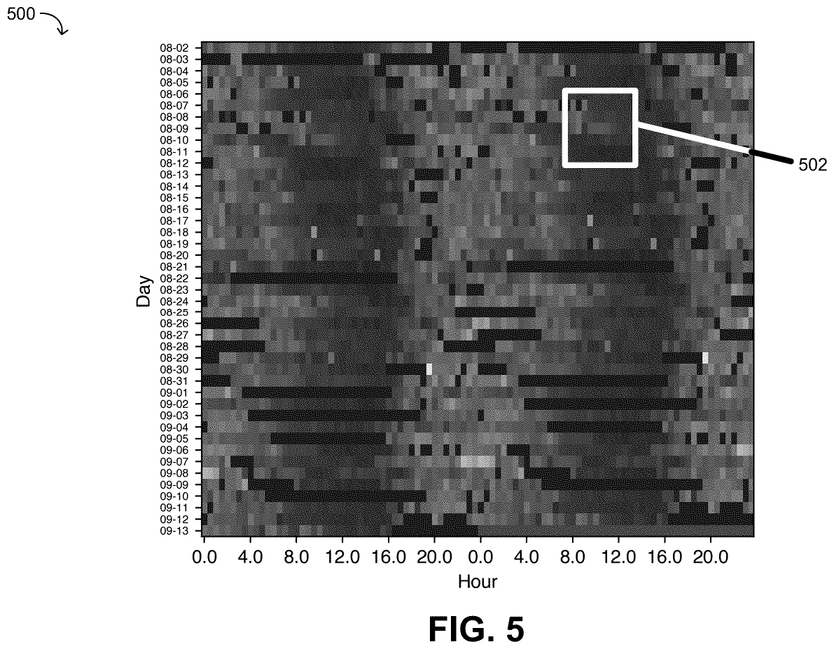

FIG. 5 illustrates another plot of calendar activity 500 as might be represented in a data structure readable by a patent interaction system and/or a patient treatment handling system. In FIG. 5, calendar activity 500 corresponds to heart rate data, with brighter rectangles relating to higher heart rate and darker rectangles relating to lower heart rate. As can be seen from the depiction of the calendar activity, there is an anomaly 502 identified in the white box. An event capturing the main activity period over the day can be extracted at the day level, represented as a date cluster. In FIG. 5, the bars of dark black rectangles represent missing data, such as where sensors did not pick up valid data. These can be filled in, or not, and filling in might be done using context or other data.

In a particular embodiment, a circadian data processing system might use biophysics models to distinguish external anomalies from internal anomalies. An internal anomaly might be related to something specific to a patient's body whereas an external anomaly might be related to something unusual happening in the patient's environment. For example, if someone gets a sustained burst of bright light exposure late at night, that can alter their circadian trajectory. A sensor system might provide information to the circadian data processing system to flag that stimuli as an external anomaly that can be unrelated to the patient. The circadian data processing system might take into account internal anomalies that are the result of external anomalies. For example, in the case of a sustained burst of bright light exposure late at night, that might cause a person's circadian trajectory to be delayed and cause them to sleep in later. By tracking both types of anomalies, the circadian data processing system can take into account internal anomalies, external anomalies, and internal anomalies likely caused by external anomalies and process the circadian trajectory accordingly.

In a specific example embodiment, the circadian data processing system might notice, perhaps via light sensors, that a user was exposed to an unusual burst of non-blued light at night, predict how much that burst would be expected to delay the user's waking up the next day, detect when the user woke up, compare the actual time to the predicted time, and if the prediction was sufficiently off, flag that as an anomaly. The circadian data processing system can thus capture internal anomalies that might not be inferred by just considering internal anomalies or just considering external anomalies.

In some implementations, a circadian data processing system might process data, detect anomalies, zero them out so they do not skew the rest of the data, identify, for each day, which wall-time corresponds to the circadian phase/state, S0 (the start of a circadian trajectory), and record how S0 moves around from day to day. This might be depicted as a curved line from top to bottom of FIG. 5 and might be referred to as a “zero-phase line.” The zero-phase line can be extrapolated to estimate wall-time to circadian phase/state mapping for a future date.

In some implementations, a circadian data processing system might filter out anomalies to process the remaining sensor data. In other implementations, the anomalies remain part of the data. This might be useful where an off-schedule day might be an anomaly for a person, which might affect their circadian trajectory and the circadian data processing system could account for that.

Some examples of anomaly pattern detection might include filling in missing data, inferring shift data for the user, emitting warnings that anomalies might cause a shift in the user's optimal timing. As examples, if a user's shift patter is normally (day shift, day shift, night shift, day off, day off) and the user misses a day of data gathering, the circadian data processing system might infer what kind of day it was for the missing data. In some cases, the user might enter their shift information but this can be inferred by the circadian data processing system.

Some users might have optimized their schedule based on their known trajectory, for example, having coffee in the morning, talking a walk in the early afternoon, doing focused work in the late afternoon, etc. The circadian data processing system can alert the user that their circadian trajectory is out of norm, such as warning them that they are likely to see a shift in their best times because they have recently had an anomalous day. For example, the circadian data processing system might issue a message like:
“You will likely feel tired in the 5 PM to 9 PM time period. You are normally alert during this period, but your circadian trajectory has shifted.”
“You likely will have trouble sleeping if you try to go to sleep at your normal time of 10 PM to 11 PM because your circadian trajectory has shifted. Try dimming lights and starting sleep before 9 PM or after midnight.”
“You likely will wake up in the middle of the night, as your body will think that 3 AM is really 8 AM.”

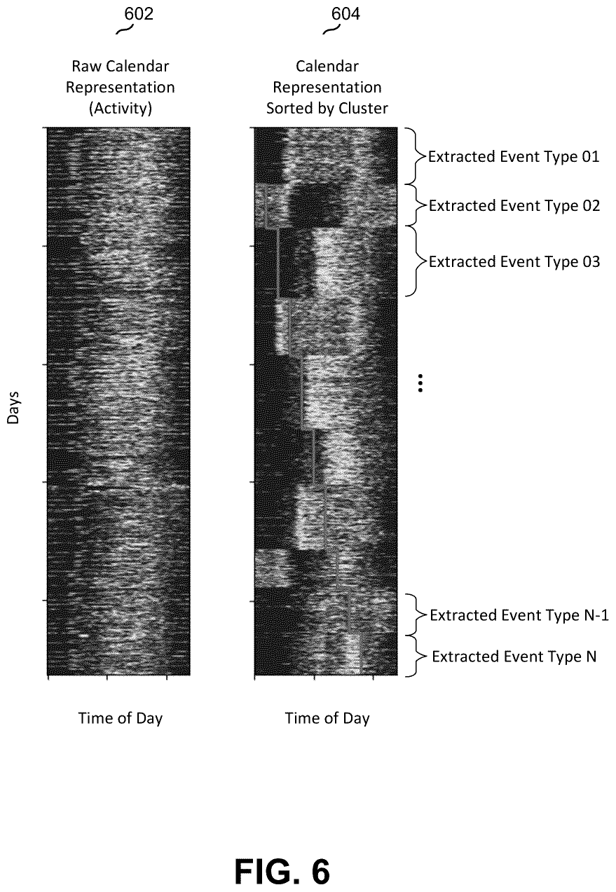

FIG. 6 illustrates a collection of day-level activity collection as might be stored in memory for use by a patient system. Data collections might represent activity over months. In the raw data collection 602, calendar activity is shown plotted sequentially by day, whereas in data collection 604, the same calendar activity data is shown but here is sorted by cluster or feature of the day's data. For example, the patient system might have a sorting unit that might perform clustering, such as using a support vector machine (SVM) model. The sorting unit might derive an activity vector for a day's data and do that for each day, then sort the day data by activity vector, which might show clusters of day-level events from long-term activity patterns. Each cluster of day-level event patterns might be labelled as being a specific event type as shown.

The data used in raw data collection 602 might be activity data generated from calendar data, user inputs, and/or body sensor data, etc. as described elsewhere herein. The patient system might use autoencoders or other clustering methods to cluster the raw data into bins each associated with a day type (more generally, an extracted event type). In a specific embodiment, the patient system collects data, such as that shown in FIGS. 4 and 5, places the data in arrays, uses SVM techniques to determine some value or characteristic for each of the arrays, sorts by that value, detects a transition from one array based on sudden changes in the value of the characteristic, and labels the arrays between two adjacent transitions to be a given event type.

Clustering and labeling events and/or days to particular “type of day” bins or extracted event types can be used for wall-time-to-circadian time mapping. For example, if there is missing data such as a pattern “AAB_AA” (the “_” representing the missing data) and a regularly occurring pattern “AABBAA” appears in similar situations, then the patient system might infer that the missing data is most likely “B.” In some embodiments, data does not have to be missing to be inferred. In some cases, after “AAB” appears, the patient system might infer “B” as the next likely move and the patient system can use that estimate to precompute its next step. In this way, a patient system might be able to precompute likely circadian trajectories for future time in advance, possibly making them load faster and render faster. Once the data is in bins or clusters, a patient system can use that structure to accelerate runtime operations, such as by simulating likely future data before the real data comes in, leaving less work to be done once the real data comes in.

In some embodiments, labeling extracted events or assigning data to bins can provide benefits, such as speeding up data entry for users, so they don't have to manually enter things that can be guessed, perhaps as a form of autocompletion, better understanding of predictors of some event, like bad sleep or bad mood (e.g., “You just had an AAB transition, so you might expect bad sleep this evening” or “You are most likely to have bad sleep after an AAB transition.”

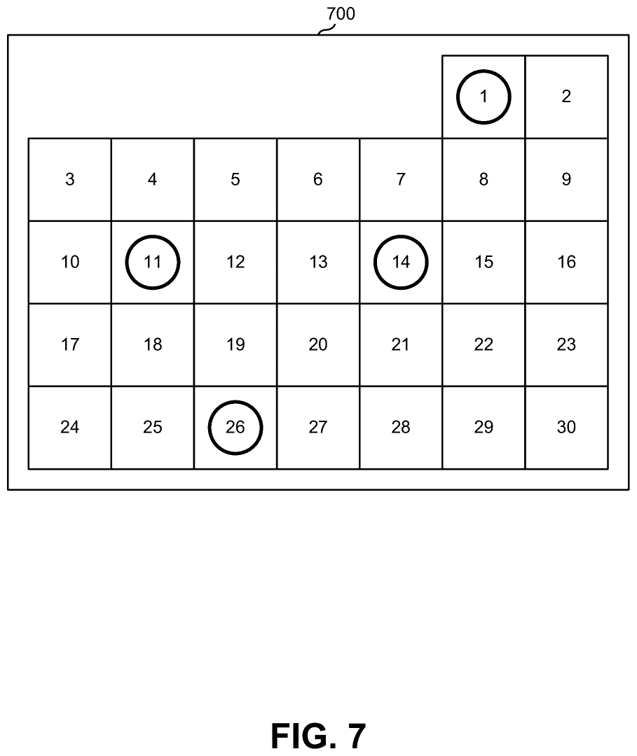

FIG. 7 illustrates an example calendar depiction 700 that might be presented to a user or used in further processing wherein calendar depiction 700 indicates which dates are clustered into which day-level event clusters. In this example, the sorting unit might have determined that, for the depicted month, the 1st, 11th, 14th, and 26th of the month were days of same “type.” It might be expected that shift workers with shift periods that vary over time might have more day-level event data clusters than a person working a more periodic schedule such as working Monday through Friday the same hours each day and having similar activity patterns each weekend day.

A user-presented depiction might be used for feedback. For example, a patent system might present a user with a proposed clustering as indicated in FIG. 7 with a message like “Here is a pattern of dates in which you had similar event patterns over the course of that day. Would you like to edit this information and/or designate these dates as being a particular type of day?” The patient system might use a user response to further refine clustering. This clustering could be used to determine a circadian state more precisely in the past, at the present, or estimated for a future time.

### Hardware Components

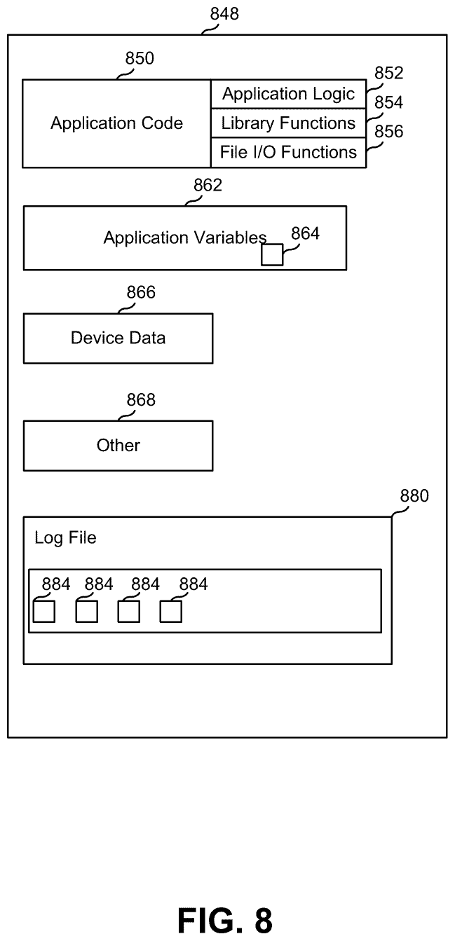

FIG. 8 is a simplified functional block diagram of a storage device 848 having an application that can be accessed and executed by a processor in a computer system as might be part of embodiments of a patient treatment system and/or a computer system that performs the computations needed for treatment optimization. FIG. 8 also illustrates an example of memory elements that might be used by a processor to implement elements of the embodiments described herein. In some embodiments, the data structures are used by various components and tools, some of which are described in more detail herein. The data structures and program code used to operate on the data structures may be provided and/or carried by a transitory computer readable medium, e.g., a transmission medium such as in the form of a signal transmitted over a network. For example, where a functional block is referenced, it might be implemented as program code stored in memory. The application can be one or more of the applications described herein, running on servers, clients or other platforms or devices and might represent memory of one of the clients and/or servers illustrated elsewhere.

Storage device 848 can be one or more memory device that can be accessed by a processor and storage device 848 can have stored thereon application code 850 that can be configured to store one or more processor readable instructions, in the form of write-only memory and/or writable memory. The application code 850 can include application logic 852, library functions 854, and file I/O functions 856 associated with the application. The memory elements of FIG. 8 might be used for a server or computer that interfaces with a user, generates data, and/or manages other aspects of a process described herein.

Storage device 848 can also include application variables 862 that can include one or more storage locations configured to receive input variables 864. The application variables 862 can include variables that are generated by the application or otherwise local to the application. The application variables 862 can be generated, for example, from data retrieved from an external source, such as a user or an external device or application. The processor can execute the application code 850 to generate the application variables 862 provided to storage device 848. Application variables 862 might include operational details needed to perform the functions described herein.

Storage device 848 can include storage for databases and other data described herein. One or more memory locations can be configured to store device data 866. Device data 866 can include data that is sourced by an external source, such as a user or an external device. Device data 866 can include, for example, records being passed between servers prior to being transmitted or after being received. Other data 868 might also be supplied.

Storage device 848 can also include a log file 880 having one or more storage locations 884 configured to store results of the application or inputs provided to the application. For example, the log file 880 can be configured to store a history of actions, alerts, error message and the like.

According to some embodiments, the techniques described herein are implemented by one or more generalized computing systems programmed to perform the techniques pursuant to program instructions in firmware, memory, other storage, or a combination. Special-purpose computing devices may be used, such as desktop computer systems, portable computer systems, handheld devices, networking devices or any other device that incorporates hard-wired and/or program logic to implement the techniques.

One embodiment might include a carrier medium carrying data that includes data having been processed by the methods described herein. The carrier medium can comprise any medium suitable for carrying the data, including a storage medium, e.g., solid-state memory, an optical disk or a magnetic disk, or a transient medium, e.g., a signal carrying the data such as a signal transmitted over a network, a digital signal, a radio frequency signal, an acoustic signal, an optical signal or an electrical signal.

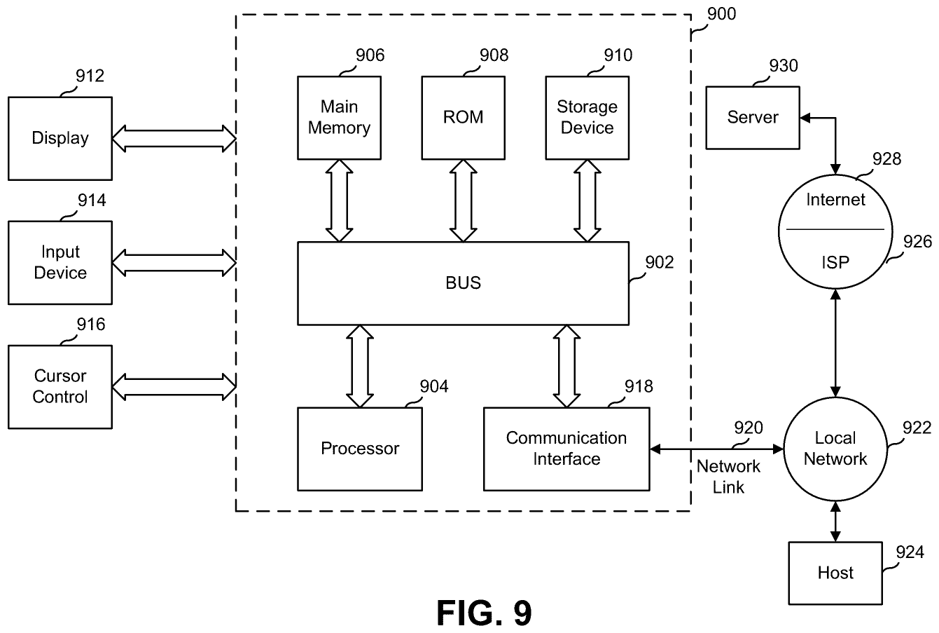

FIG. 9 is a block diagram that illustrates a computer system 900 upon which the computer systems of the systems described herein and/or data structures shown in various other figures, such as FIG. 8, may be implemented. Computer system 900 includes a bus 902 or other communication mechanism for communicating information, and a processor 904 coupled with bus 902 for processing information. Processor 904 may be, for example, a general-purpose microprocessor.

Computer system 900 also includes a main memory 906, such as a random-access memory (RAM) or other dynamic storage device, coupled to bus 902 for storing information and instructions to be executed by processor 904. Main memory 906 may also be used for storing temporary variables or other intermediate information during execution of instructions to be executed by processor 904. Such instructions, when stored in non-transitory storage media accessible to processor 904, render computer system 900 into a special-purpose machine that is customized to perform the operations specified in the instructions.

Computer system 900 further includes a read only memory (ROM) 908 or other static storage device coupled to bus 902 for storing static information and instructions for processor 904. A storage device 910, such as a magnetic disk or optical disk, is provided and coupled to bus 902 for storing information and instructions.

Computer system 900 may be coupled via bus 902 to a display 912, such as a computer monitor, for displaying information to a computer user. An input device 914, including alphanumeric and other keys, is coupled to bus 902 for communicating information and command selections to processor 904. Another type of user input device is a cursor control 916, such as a mouse, a trackball, or cursor direction keys for communicating direction information and command selections to processor 904 and for controlling cursor movement on display 912. This input device typically has two degrees of freedom in two axes, a first axis (e.g., x) and a second axis (e.g., y), that allows the device to specify positions in a plane.

Computer system 900 may implement the techniques described herein using customized hard-wired logic, one or more ASICs or FPGAs, firmware and/or program logic which in combination with the computer system causes or programs computer system 900 to be a special-purpose machine. According to one embodiment, the techniques herein are performed by computer system 900 in response to processor 904 executing one or more sequences of one or more instructions contained in main memory 906. Such instructions may be read into main memory 906 from another storage medium, such as storage device 910. Execution of the sequences of instructions contained in main memory 906 causes processor 904 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may include non-volatile media and/or volatile media. Non-volatile media includes, for example, optical or magnetic disks, such as storage device 910. Volatile media includes dynamic memory, such as main memory 906. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, an EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire, and fiber optics, including the wires that include bus 902. Transmission media can also take the form of acoustic or light waves, such as those generated during radio-wave and infra-red data communications.

Various forms of media may be involved in carrying one or more sequences of one or more instructions to processor 904 for execution. For example, the instructions may initially be carried on a magnetic disk or solid-state drive of a remote computer. The remote computer can load the instructions into its dynamic memory and send the instructions over a network connection. A modem or network interface local to computer system 900 can receive the data. Bus 902 carries the data to main memory 906, from which processor 904 retrieves and executes the instructions. The instructions received by main memory 906 may optionally be stored on storage device 910 either before or after execution by processor 904.

Computer system 900 also includes a communication interface 918 coupled to bus 902. Communication interface 918 provides a two-way data communication coupling to a network link 920 that is connected to a local network 922. For example, communication interface 918 may be a network card, a modem, a cable modem, or a satellite modem to provide a data communication connection to a corresponding type of telephone line or communications line. Wireless links may also be implemented. In any such implementation, communication interface 918 sends and receives electrical, electromagnetic, or optical signals that carry digital data streams representing various types of information.

Network link 920 typically provides data communication through one or more networks to other data devices. For example, network link 920 may provide a connection through local network 922 to a host computer 924 or to data equipment operated by an Internet Service Provider (ISP) 926. ISP 926 in turn provides data communication services through the world-wide packet data communication network now commonly referred to as the “Internet” 928. Local network 922 and Internet 928 both use electrical, electromagnetic, or optical signals that carry digital data streams. The signals through the various networks and the signals on network link 920 and through communication interface 918, which carry the digital data to and from computer system 900, are example forms of transmission media.

Computer system 900 can send messages and receive data, including program code, through the network(s), network link 920, and communication interface 918. In the Internet example, a server 930 might transmit a requested code for an application program through the Internet 928, ISP 926, local network 922, and communication interface 918. The received code may be executed by processor 904 as it is received, and/or stored in storage device 910, or other non-volatile storage for later execution.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory. The code may also be provided carried by a transitory computer readable medium e.g., a transmission medium such as in the form of a signal transmitted over a network.

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., may be either A or B or C, or any nonempty subset of the set of A and B and C. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present.

The use of examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

Further embodiments can be envisioned to one of ordinary skill in the art after reading this disclosure. In other embodiments, combinations or sub-combinations of the above-disclosed invention can be advantageously made. The example arrangements of components are shown for purposes of illustration and combinations, additions, re-arrangements, and the like are contemplated in alternative embodiments of the present invention. Thus, while the invention has been described with respect to exemplary embodiments, one skilled in the art will recognize that numerous modifications are possible.

For example, the processes described herein may be implemented using hardware components, software components, and/or any combination thereof. The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

All references, including publications, patent applications, and patents, cited herein are hereby incorporated by reference to the same extent as if each reference were individually and specifically indicated to be incorporated by reference and were set forth in its entirety herein.

What is claimed is:

1. A computer-implemented method for processing an image to extract data, comprising:
under control of one or more computer systems configured with executable instructions:
obtaining a digital representation of the image;
determining a context for elements in the digital representation;
determining one or more calendar items among the elements in the digital representation;
generating at least one event record for a calendar item, where in the event record comprises a start date data field, an end date data field, a start time data field, and an end time data field; and
providing the at least one event record to a patient system in a form usable for determining a circadian trajectory.

2. The computer-implemented method of claim 1, wherein the digital representation includes a grid pattern representing dates of a calendar.

3. The computer-implemented method of claim 1, wherein the at least one event record further comprises an event category and a textual event descriptor.

4. The computer-implemented method of claim 1, wherein at least a first event record of the at least one event record represents a positive event that is scheduled to occur based on the digital representation and wherein at least a second event record of the at least one event record represents a negative event indicative of a time per within a calendar span in which activity is not occurring.

5. The computer-implemented method of claim 1, further comprising segmenting the digital representation into a plurality of day units.

6. The computer-implemented method of claim 5, further comprising organizing the plurality of day units into one or more date clusters, wherein at least one date cluster comprises a plurality of dates over which an event is deemed to occur.

7. The computer-implemented method of claim 6, further comprising determining from the at least one date cluster a historical pattern of event data.

8. The computer-implemented method of claim 1, further comprising determining preferred shift combinations for an individual associated with a calendar represented in the digital representation.

9. The computer-implemented method of claim 1, wherein the circadian trajectory is derived by a scheduler using at least one biophysics model of a human circadian clock and at least one a statistical model of a human circadian clock.

10. A non-transitory computer-readable storage medium storing instructions, which when executed by at least one processor of a computer system, causes the computer system to carry out the method of any one of claims 1 to 9.

11. A computer system comprising:
one or more processors; and
a storage medium storing instructions, which when executed by the one or more processors, cause the computer system to implement the method of any one of claims 1 to 9.

12. A computer-implemented method for determining a circadian state of a person, comprising:
under control of one or more computer systems configured with executable instructions:
a) obtaining user data over a plurality of days, the user data related to the person's body;
b) determining, for each of at least a subset of the plurality of days, an event type for each day;
c) determining a circadian trajectory for a past day of the plurality of days;
d) estimating a circadian trajectory and/or a circadian state for a future day and a mapping of the circadian trajectory and/or the circadian state to wall-clock time; and
e) using the mapping to determine when to initiate or announce a biological treatment for the person based on a preference of circadian state for the biological treatment.

13. The computer-implemented method of claim 12, wherein the user data comprises computer-readable data representing user planned or actual activity.

14. The computer-implemented method of claim 13, wherein the user planned or actual activity is extracted from a calendar.

15. The computer-implemented method of claim 12, wherein the user data comprises data from sensors that sense bodily actions, quantities, or functions from the person's body.

16. The computer-implemented method of claim 15, wherein the sensors are wearable devices on the person's body.

17. The computer-implemented method of claim 12, wherein estimating the circadian trajectory and/or the circadian state comprises filtering the user data to reduce effects of anomalies and/or missing data.
